(19) United States
(12) Patent Application Publication
(10) Pub. No.: US 2012/0070900 A1
(43) Pub. Date: Mar. 22, 2012

(54) T-DNA/PROTEIN NANO-COMPLEXES FOR PLANT TRANSFORMATION

(75) Inventors: Igor KOVALCHUK, Lethbridge (CA); Alicja ZIEMIENOWICZ, Lethbridge (CA); Francois EUDES, Lethbridge (CA)

(73) Assignee: PLANTBIOSIS LTD., Lethbridge (CA)

(21) Appl. No.: 13/152,163

(22) Filed: Jun. 2, 2011

Related U.S. Application Data
(60) Provisional application No. 61/350,774, filed on Jun. 2, 2010.

(51) Int. Cl. C12N 15/82; C12N 9/96; B82Y 99/00

(52) U.S. Cl. 435/468; 435/188; 977/795; 977/902

(57) ABSTRACT

A DNA/protein nano-complex for transfecting eukaryotic cells, wherein the nano-complex comprises an expressible nucleotide molecule complexed with a VirD2 protein molecule, and a RecA protein molecule complexed with the nucleotide molecule/VirD2 protein molecule complex. The nucleotide molecule/VirD2 protein molecule/RecA protein molecule complexed may be further complexed to a cell penetrating peptide. The DNA/protein nano-complex can be used for delivering the expressible nucleotide molecule to target plant cells, in particular, monocot plant cells exemplified by wheat cells, corn cells, triticale cells, barley cells, and rye cells.

### TECHNICAL FIELD

The present disclosure relates to transformation of plant cells. In particular, the present disclosure relates to a DNA/protein nano-complex comprising an expressible nucleotide sequence for transforming plant cells. The present disclosure also pertains to methods for preparing the DNA/protein nano-complex, and to methods for transforming plant cells.

### BACKGROUND

Transgenesis, also referred to as genetic transformation, allows for the generation of plants with improved traits significantly faster than any conventional breeding practice. This technology is based on the delivery of genes of interest from a broad range of sources into a plant genome. Two major transformation techniques include Agrobacterium-mediated DNA delivery and biolistic DNA transfer. Agrobacterium-mediated transformation relies on the ability of Agrobacterium tumefaciens to transfer a portion of its DNA, called transferred DNA or T-DNA, into plant cells. During its transit from the bacterial cell to the plant nucleus, the single-stranded T-DNA is protected by a single-stranded binding protein (VirE2) and guided by the VirD2 protein. The latter is important for bringing the T-DNA into the nucleus and possibly for integrating it into the genome. The integration process apparently requires broken DNA or at least an area of active replication or transcription. Agrobacterium-mediated transformation is an efficient process and typically in dicotyledonous (dicots) plants predominantly results in integration of the transgenes at single locus; integrated T-DNA is mostly intact and allows normal expression of the transgene. However, transformation of monocotyledonous (monocots) plants with Agrobacterium tumefaciens is not very efficient. This may be due to inability of Agrobacteria to efficiently attach to the cell wall of monocots. This creates substantial problems, since quite a number of important agricultural crops are monocots (wheat, corn, triticale, barley, rye etc.). In addition, most of the vectors used for Agrobacterium-mediated transformation have common vector backbones and thus are frequent targets of rearrangements occurring prior to integration. This creates complex transgene integration patterns.

An alternative method used for transforming monocots is based on simple gold-particle mediated bombardment of naked DNA into plant tissue. The DNA is not protected against endonucleases during such biolistic transformation and the technique relies on host import proteins to transfer the DNA inside the nucleus. Hence biolistic transformation is inefficient and may generate multiple integrations of truncated, duplicated and/or rearranged transgenes.

It has been suggested that a specifically designed DNA/protein complex may be used for transforming plant cells (e.g., WO 95/05471) or animal cells (e.g., U.S. Pat. No. 6,498,011). The complex contains a chimeric recombinant DNA construct covalently associated with a VirD2 protein. The complex can be accompanied by further Vir proteins such as VirE2. It has been suggested that VirE2 may aid in the transfer of the complex through the plant cell plasma membrane (2001, Dumas F. et al., An Agrobacterium VirE2 channel for transferred-DNA transport into plant cells. Proc. Natl. Acad. Sci. USA, 98: 485-490).

While a DNA/VirD2 complex might be able deliver a DNA molecule to the nucleus of a target cell, the complex is not protected from, for example, endonucleases in the cytoplasm. In Agrobacterium-mediated transformations, the DNA is protected by VirE2 and, hence, it has been suggested to add this protein to the complex. However, for a variety of reasons, VirE2 is difficult to purify in useful quantities.

### SUMMARY

The present disclosure provides novel T-DNA/protein nano-complexes useful for transforming plant cells. The T-DNA/protein nano-complexes can be prepared in vitro. The nano-complexes protect associated T-DNA molecules from degradation during and after delivery to target plant cells. The present disclosure further provides a method of transforming plant cells using the T-DNA/protein nano-complexes. The present approach may lead to integration of fewer and more intact copies of delivered DNA molecules. The present approach may be a suitable alternative to the bombardment techniques currently used for monocots.

This summary does not necessarily describe all features of the invention. Other aspects, features and advantages of the invention will be apparent to those of ordinary skill in the art upon review of the following description of specific embodiments of the invention.

### BRIEF DESCRIPTION OF THE DRAWINGS

FIG. 1 is a flowchart illustrating the construction of an exemplary DNA/VirD2/RecA nano-complex of the present invention;

FIG. 2 shows an exemplary experimental procedure for producing transgenic plants from cells transfected with the exemplary DNA/VirD2/RecA nano-complex from FIG. 1;

FIG. 3 shows the structure of an exemplary T-DNA/protein nano-complex of the present invention; containing the GUS reporter gene;

FIG. 4 shows the scheme of the GUS gene expression cassette in constructs used in transfection experiments: 4(a) shows the 4.8 kb long T-DNA, and 4(b) shows the pACT-1D/PstI construct;

FIG. 5(a) is a micrograph of a gel showing the cleavage activity of the recombinant VirD2 protein in the presence or absence of 1 μg of VirD2, and FIG. 5(b) is a micrograph of a gel showing the activity of the RecA protein in a reaction mixture containing TKM buffer and various amounts of RecA protein;

FIG. 6 shows micrographs depicting the development of embryos from microspores treated with various DNAs in the presence or absence of Tat₂ peptide;

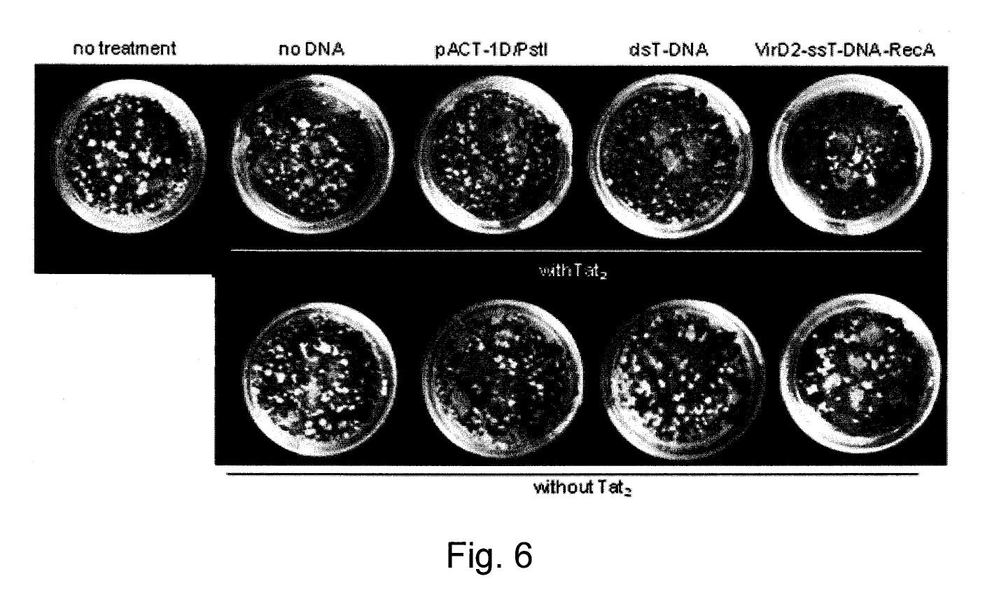

FIG. 7 is a micrograph showing regeneration of embryos into green plantlets, albino plantlets, rooted embryos and aborted embryos;

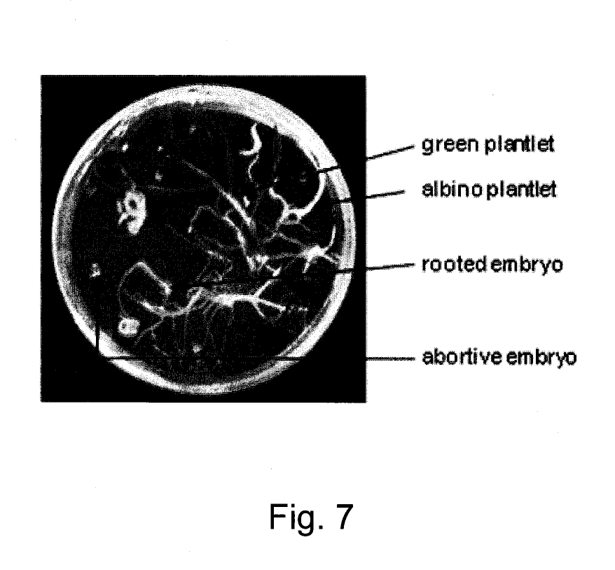

FIG. 8 shows micrographs of southern blot analysis of transgenic triticale plants for detection of the GUS transgene in gDNA of GUS-PCR-positive plants;

FIG. 9 shows integration patterns of T-DNA containing GUS expression cassette isolated from leaf samples collected from transgenic triticale plants;

FIG. 10 are micrographs of gels produced by Western blotting to assess transgene expression in protein extracts isolated from GUS-positive plants regenerated from triticale microspores treated with various DNAs (dsT-DNA, ssT-DNA) or DNA/protein complexes (ssT-DNA-RecA, VirD2-ssT-DNA, VirD2-ssT-DNA-RecA) in the presence of the Tat₂ peptide; and

FIG. 11 is a micrograph of a gel produced by the southern blot technique to determine the transgene copy number and integration patterns in transgenic triticale plants regenerated from microspores transfected with the nano-complex (VirD2-ssT-DNA-RecA+Tat₂).

### DETAILED DESCRIPTION

Unless otherwise defined, all technical and scientific terms used herein have the same meaning as commonly understood by one of ordinary skill in the art to which this invention belongs. In order that the invention herein described may be fully understood, the following terms and definitions are provided herein.

As used herein, the term “synthetic DNA” means DNA sequences that have been prepared entirely or at least partially by chemical means. Synthetic DNA sequences may be used, for example, for modifying native DNA sequences in terms of codon usage and expression efficiency.

The word “comprise” or variations such as “comprises” or “comprising” will be understood to imply the inclusion of a stated integer or groups of integers but not the exclusion of any other integer or group of integers.

As used herein, the word “complexed” means attached together by one or more linkages.

The term “a cell” includes a single cell as well as a plurality or population of cells.

The term “about” or “approximately” means within 20%, preferably within 10%, and more preferably within 5% of a given value or range.

The term “nucleic acid” refers to a polymeric compound comprised of covalently linked subunits called nucleotides. Nucleic acid includes polyribonucleic acid (RNA) and polydeoxyribonucleic acid (DNA), both of which may be single-stranded or double-stranded. DNA includes cDNA, genomic DNA, synthetic DNA, and semisynthetic DNA.

The term “gene” refers to an assembly of nucleotides that encode a polypeptide, and includes cDNA and genomic DNA nucleic acids.

The term “recombinant DNA molecule” refers to a DNA molecule that has undergone a molecular biological manipulation.

The term “vector” refers to any means for the transfer of a nucleic acid into a host cell. A vector may be a replicon to which another DNA segment may be attached so as to bring about the replication of the attached segment. A “replicon” is any genetic element (e.g., plasmid, phage, cosmid, chromosome, virus) that functions as an autonomous unit of DNA replication in vivo, i.e., capable of replication under its own control. The term “vector” includes plasmids, DNA-protein complexes, and biopolymers. In addition to a nucleic acid, a vector may also contain one or more regulatory regions, and/or selectable markers useful in selecting, measuring, and monitoring nucleic acid transfer results (transfer to which tissues, duration of expression, etc.).

The term “cloning vector” refers to a replicon, such as plasmid, phage or cosmid, to which another DNA segment may be attached so as to bring about the replication of the attached segment. Cloning vectors may be capable of replication in one cell type, and expression in another (“shuttle vector”).

A cell has been “transfected” by exogenous or heterologous DNA when such DNA has been introduced inside the cell. A cell has been “transformed” by exogenous or heterologous DNA when the transfected DNA effects a phenotypic change. The transforming DNA can be integrated (covalently linked) into chromosomal DNA making up the genome of the cell.

The term “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms.

Modification of a genetic and/or chemical nature is understood to mean any mutation, substitution, deletion, addition and/or modification of one or more residues. Such derivatives may be generated for various purposes, such as in particular that of enhancing its production levels, that of increasing and/or modifying its activity, or that of conferring new pharmacokinetic and/or biological properties on it. Among the derivatives resulting from an addition, there may be mentioned, for example, the chimeric nucleic acid sequences comprising an additional heterologous part linked to one end, for example of the hybrid construct type consisting of a cDNA with which one or more introns would be associated.

Likewise, for the purposes of the invention, the claimed nucleic acids may comprise promoter, activating or regulatory sequences, and the like.

The term “promoter sequence” refers to a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background.

A coding sequence is “under the control” of transcriptional and translational control sequences in a cell when RNA polymerase transcribes the coding sequence into mRNA, which is then trans-RNA spliced (if the coding sequence contains introns) and translated into the protein encoded by the coding sequence.

The term “homologous” in all its grammatical forms and spelling variations refers to the relationship between proteins that possess a “common evolutionary origin,” including homologous proteins from different species. Such proteins (and their encoding genes) have sequence homology, as reflected by their high degree of sequence similarity. This homology is greater than about 75%, greater than about 80%, greater than about 85%. In some cases the homology will be greater than about 90% to 95% or 98%.

“Amino acid sequence homology” is understood to include both amino acid sequence identity and similarity. Homologous sequences share identical and/or similar amino acid residues, where similar residues are conservative substitutions for, or “allowed point mutations” of, corresponding amino acid residues in an aligned reference sequence. Thus, a candidate polypeptide sequence that shares 70% amino acid homology with a reference sequence is one in which any 70% of the aligned residues are either identical to, or are conservative substitutions of, the corresponding residues in a reference sequence.

The term “polypeptide” refers to a polymeric compound comprised of covalently linked amino acid residues. Amino acids are classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group. A polypeptide of the invention preferably comprises at least about 14 amino acids.

The term “protein” refers to a polypeptide which plays a structural or functional role in a living cell.

The term “VirD2” refers to the VirD2 protein which is useful for integrating T-DNA into plant genomes as described by Ziemienowicz et al. (2008, Mechanisms of T-DNA integration. In: Tzfira et al. (Eds.) Agrobacterium: from biology to biotechnology. pp 396-441. Springer, New York, USA).

The term “VirE2” refers to the VirE2 protein which protects single-stranded T-DNA during transfer of the T-DNA from microbial cells to plant nuclei as described by Rossi et al. (1996, Integration of complete transferred DNA units is dependent on the activity of virulence E2 protein of Agrobacterium tumefaciens. Proc. Natl. Acad. Sci. USA, 93:126-130).

The term “corresponding to” is used herein to refer to similar or homologous sequences, whether the exact position is identical or different from the molecule to which the similarity or homology is measured. A nucleic acid or amino acid sequence alignment may include spaces. Thus, the term “corresponding to” refers to the sequence similarity, and not the numbering of the amino acid residues or nucleotide bases.

The term “derivative” refers to a product comprising, for example, modifications at the level of the primary structure, such as deletions of one or more residues, substitutions of one or more residues, and/or modifications at the level of one or more residues. The number of residues affected by the modifications may be, for example, from 1, 2 or 3 to 10, 20, or 30 residues. The term derivative also comprises the molecules comprising additional internal or terminal parts, of a peptide nature or otherwise. They may be in particular active parts, markers, amino acids, such as methionine at position −1. The term derivative also comprises the molecules comprising modifications at the level of the tertiary structure (N-terminal end, and the like). The term derivative also comprises sequences homologous to the sequence considered, derived from other cellular sources, and in particular from cells of human origin, or from other organisms, and possessing activity of the same type or of substantially similar type. Such homologous sequences may be obtained by hybridization experiments. The hybridizations may be performed based on nucleic acid libraries, using, as probe, the native sequence or a fragment thereof, under conventional stringency conditions or preferably under high stringency conditions.

The embodiments of the present invention relate to novel T-DNA/protein nano-complexes useful for delivering selected DNA molecules to target cells for the purpose of transforming the cells, and to methods for preparing the novel T-DNA/protein nano-complexes. The selected DNA may be either of homologous or heterologous origin with respect to the plant material involved or it may be of synthetic origin or both. The DNA sequence can be constructed from genomic DNA, from cDNA, from synthetic DNA, or hybrids thereof.

The DNA may be single-stranded. VirD2 is able to cleave single-stranded DNA. For processing double-stranded DNA (e.g. plasmids) additional proteins may be necessary such as those exemplified by VirD1.

The DNA may comprise a recognition sequence for VirD2. For example, the DNA may comprise the so called right border (RB) sequence from Agrobacterium pTi plasmid as disclosed by Ziemienowicz et al. (2000, Plant enzymes but not Agrobacterium VirD2 mediate T-DNA ligation in vitro. Mol. Cell. Biol. 20: 6317-6322).

The DNA preferably comprises the RB sequence which is 24 nucleotides in length (although shorter oligonucleotides (e.g. 17 nt) can be processed by VirD2. DNAs carrying the RB sequence are named hereinafter as “T-DNA”.

The DNA may be of any suitable size. For example, the DNA may be 10-6500 bases long, or 15-5000 bases long, or 25-4500 bases long, or 100-2500 bases long. In theory, the DNA may be up to 200,000 bases long, such as the entire pTi plasmid of Agrobacterium (T-DNA in the pTi plasmid is 20,000 by long). However, while not wishing to be bound by theory, it is believed that the cleavage efficiency is reduced for longer molecules.

Suitable for use herein is virtually any DNA composition that may be delivered to plant cells to ultimately produce fertile transgenic plants. For example, regulatory elements such as plant promoters; a sequence that acts as a signal to terminate transcription and allow for the poly-adenylation of the resultant mRNA; or a specific leader sequence which may, for example, increase or maintain mRNA stability and prevent inappropriate initiation of translation. It may be desirable to introduce DNA for genes or gene families which encode a desired traits for agricultural crops such as, but not limited to, herbicide resistance or tolerance (e.g. glycophosphate-resistance genes); insect resistance or tolerance; disease resistance or tolerance (viral, bacterial, fungal, nematode); stress tolerance and/or resistance, as exemplified by resistance or tolerance to drought, heat, chilling, freezing, excessive moisture, salt stress; oxidative stress; increased yields; food content and makeup; physical appearance; male sterility; drydown; standability; prolificacy; starch properties; oil quantity and quality; and the like. One may desire to incorporate one or more genes conferring any such desirable trait or traits, such as, for example, a gene or genes encoding herbicide resistance.

DNA may be introduced for the purpose of expressing RNA transcripts that function to affect plant phenotype yet are not translated into protein. Two examples are antisense RNA and RNA with ribozyme activity. Both may serve possible functions in reducing or eliminating expression of native or introduced plant genes.

DNA may be introduced for other purposes. For example, DNA elements including those of transposable elements such as Ds, Ac, or Mu, may be inserted into a gene and cause mutations. These DNA elements may be inserted in order to inactivate (or activate) a gene and thereby “tag” a particular trait.

In certain embodiments, the present invention contemplates the transformation of a recipient cell with more than one advantageous transgene. Two or more transgenes can be supplied in separate vectors, or alternatively, in a single vector that incorporates two or more gene coding sequences.

The T-DNA/protein nano-complexes of the present invention comprise VirD2. This protein is known to have a nuclear targeting function and can deliver ssDNA to the nucleus. However, when VirD2 is used alone, the DNA is unprotected from endonuclease activity which reduces the likelihood of a successful transformation. Furthermore, it is believed that VirD2 cannot aid in penetration of the complex through plant cell walls.

Suitable homologs of VirD2 may be used such as those exemplified by TraI from E. coli (Pansegrau et al., 1993, Site-specific cleavage and joining of single-stranded DNA by VirD2 protein of Agrobacterium tumefasciens Ti plasmids: Analogy to bacterial conjugation. Proc. Natl. Acad. Sci. USA 90: 11538-11542). The VirD2 may be obtained by any suitable means. For example, the protein may be purified in accordance with the method taught by Ziemienowicz et al. (2001, Import of Agrobacterium T-DNA into plant nuclei: two distinct functions of VirD2 and VirE2 proteins. Plant Cell 13: 369-383).

The T-DNA/protein nano-complexes of the present invention comprise RecA, a protein isolated from Escherichia coli. RecA has a DNA-repair and maintenance function in E. coli. RecA is relatively easy to isolate and is available commercially (e.g., New England Biolabs, Ipswich, Mass., USA; Bio-Concept Laboratories Inc., Salem, N.H., USA). It has been suggested that RecA could serve as a substitute for the nuclear import function of VirE2 but that RecA cannot substitute for VirE2 in efficient T-DNA transfer (Ziemienowicz et al., 2001, Plant Cell 13: 369-383).

Surprisingly, despite the prior art teachings, it has been found that the T-DNA/protein nano-complexes of the present invention comprising RecA can efficiently transform plant cells.

Various homologs of RecA are known and may be used herein. For example, Tth RecA, yeast Rad51, or any other single-strand DNA-binding protein of prokaryotic or eukaryotic origin which forms filaments with similar structure as ssDNA-RecA filaments.

The present complex may be delivered to the target cells by any suitable means. Such techniques are known in the art and are exemplified by electroporation, bombardment, microinjection, liposomes, and the like.

The T-DNA/protein nano-complexes of the present invention may additionally comprise a compatible cell penetrating peptide exemplified by Tat2. Cell penetrating peptides (CPP) are a class of relatively short peptides that have the ability to translocate across cell membranes. Any suitable CPP may be used herein. For example, the T-DNA/protein nano-complexes may comprise a Tat2 peptide having an amino acid sequence RKKRRQRRRRKKRRQRRR (SEQ ID NO: 1). An alternative CPP system is described in U.S. Pat. No. 6,841,535. CPPs may be obtained from companies offering peptide synthesis service (e.g., Biomatik Corp., Cambridge, ON, Canada; Pacific Immunology Corpo, Ramona, Calif., USA; LifeTein LLC, South Plainfield, N.J., USA). Some CPPs are available in a form of kits (e.g., the Chariot Protein Delivery Kit from Active Motif, Carlsbad, Calif., USA).

While not wishing to be bound by theory, it is believed that the single stranded T-DNA molecule is covalently linked to VirD2. Then, the T-DNA/VirD2 complex is covered by RecA, a protein with high affinity for single stranded DNA. The ssT-DNA/VirD2/RecA complex is then linked to CPP in vitro, forming a nano-complex that has the ability to transfect plant cells. The CPP is believed to release the ssT-DNA/VirD2/RecA complex in the cytoplasm, and VirD2 guides the complex to the nucleus. The ssT-DNA is passively protected by RecA from nuclease activity in the cytoplasm. Upon reaching the nucleus, RecA protein dissociates and VirD2 assists the T-DNA in its integration into the genome.

The T-DNA/protein nano-complexes of the present invention may be assembled in any suitable manner known to those skilled in these arts. An exemplary process for assembling an exemplary T-DNA/protein nano-complex comprises the following steps:
Step 1: preparing a DNA construct that comprises an expressible DNA sequence in operable linkage with a VirD2 target sequence;
Step 2: exposing the DNA construct in vitro to VirD2 such that target sequence is cleaved and a DNA/VirD2 complex is formed;
Step 3: exposing the DNA/VirD2 complex to RecA such that a DNA/VirD2/RecA complex is formed; and
Step 4: optionally exposing the DNA/protein complex to a cell penetrating peptide such that a DNA/VirD2/RecA/CPP complex is formed.

The T-DNA/protein nano-complexes of the present invention may be used to transform any suitable plant cell target. For example, cells from angiosperms (dicots, monocots). The present complex may be particularly useful for transforming cells from monocots such as wheat, corn, triticale, barley, rye and the like.

The present T-DNA/protein nano-complexes may be delivered, for example, to spores derived from monocots, for example wheat microspores, corn microspores, triticale microspores, barley microspores, rye microspores, and the like. The greatest advantage of microspore regeneration is the ability to obtain double haploids and thus to faster obtain plants homozygous for the transgene than is possible with standard methods currently used by those skilled in these arts. Use of the T-DNA/protein nano-complexes of the present invention to transform target plant cells enables skipping an entire generation and the lengthy and costly routine of selection for the homozygous lines.

The transformation may be performed in any suitable manner known to those skilled in these arts. An exemplary process for transformation comprises the following steps:

Step 1: preparing a T-DNA/protein nano-complex according to the present disclosure; and

Step 2: exposing target plant cells to the T-DNA/protein nano-complex.

The present method may have a transformation efficiency of about 1% or greater, about 2% or greater, about 5% or greater, about 7% or greater, about 10% or greater, about 15% or greater, about 17% or greater, about 20% or greater, about 22% or greater, about 25% or greater. The efficiency of transformation may be calculated by dividing the number transgenic plants by the number of plants regenerated.

All citations are herein incorporated by reference, as if each individual publication was specifically and individually indicated to be incorporated by reference herein and as though it were fully set forth herein. Citation of references herein is not to be construed nor considered as an admission that such references are prior art to the present invention.

The invention includes all embodiments, modifications and variations substantially as hereinbefore described and with reference to the examples and figures. It will be apparent to persons skilled in the art that a number of variations and modifications can be made without departing from the scope of the invention as defined in the claims. Examples of such modifications include the substitution of known equivalents for any aspect of the invention in order to achieve the same result in substantially the same way.

### EXAMPLES

### Example 1

### Preparation of an Exemplary T-DNA/Protein Nano-Complex

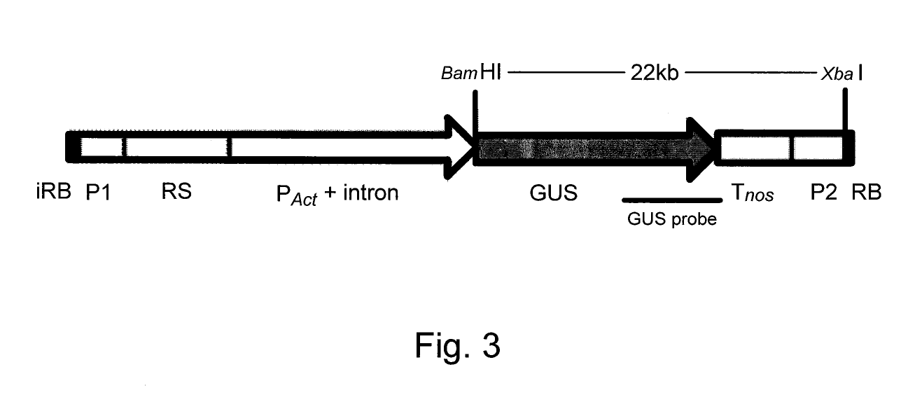

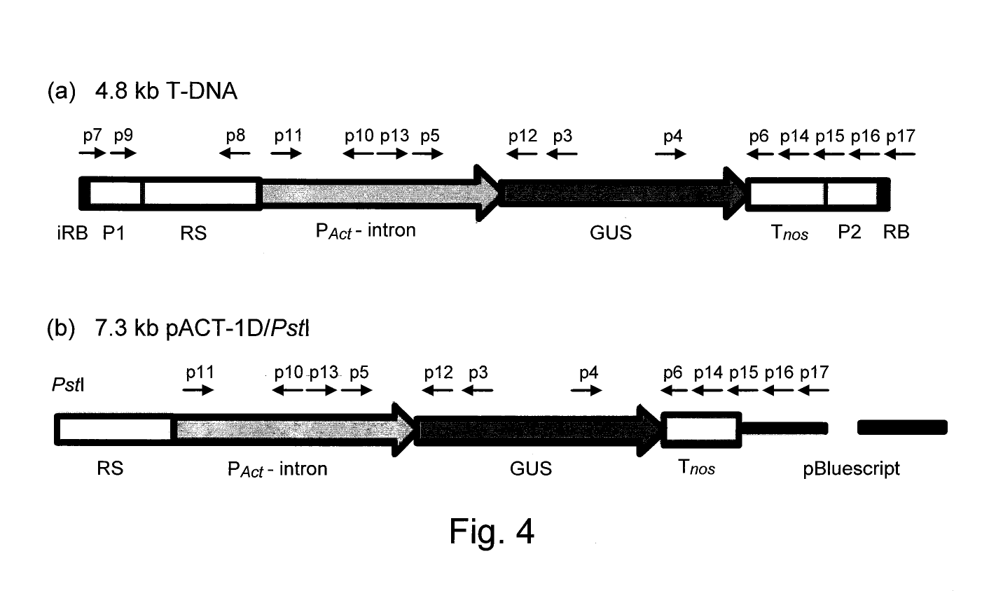

To form an exemplary T-DNA/protein nano-complex in vitro, a GUS expression cassette consisting of rice Actin promoter and intron, uidA (GUS) gene and nos terminator was amplified by PCR using primers annealing 200 by upstream (forward primer) and downstream (reverse primer) in a pACT-1D plasmid. The primers were designed to contain the Agrobacterium right border sequence (RB) in direct and inverted orientation at the 3′ and 5′ end of the GUS cassette, respectively (FIGS. 3 and 4). Such design allows for both DNA strands to serve as T-DNA, thus maximizing the use of each DNA molecule. As described in more detail below, the primers contained the NcoI restriction site for convenient cloning of the amplified iRB_P_{Act}-GUS-Tₙₒₜ_{_{—}}RB insert into Litmus29 vector. The recombinant vector was then used to produce dsT-DNA in high quantities. dsT-DNA was converted into the single stranded form (ssT-DNA) by heat denaturation. Formation of the T-DNA/protein complexes was achieved by reacting ssT-DNA with the purified recombinant proteins VirD2 and RecA and the reaction efficiency was monitored following the method taught by Ziemienowicz et al. (1999, Import of DNA into mammalian nuclei by proteins originating from a plant pathogenic bacterium. Proc. Natl. Acad. Sci. USA 96: 3729-3733). A suitable VirD2 protein will have at least 75% homology with SEQ ID NO: 22. A suitable RecA protein will have at least 75% homology with SEQ ID NO: 23.

Agrobacterium VirD2 protein was purified following the methods taught by Ziemienowicz et al. (2001). The recombinant VirD2 protein was produced in E. coli as 6×His fusion, and purified using affinity and ion-exchange chromatography following the method disclosed by Pelczar et al. (2004, Agrobacterium proteins VirD2 and VirE2 mediate precise integration of synthetic T-DNA complexes in mammalian cells. EMBO Rep. 5: 632-637).

ssT-DNA containing the Act_GUS_nos cassette was prepared generally following the methods taught by Ziemienowicz et al. (1999,) and Ziemienowicz et al. (2001). The DNA insert was produced by PCR using primers p1 and p2 containing RB sequence and NcoI site (Table 1) and pACT-1D plasmid as a template.

TABLE 1

| SEQ ID NO: | Primer | Sequence |
|---|---|---|
| SEQ ID NO: 2 | p1 | AGCCATGGTATATATCCTG\hat{ }CCACTCTTCGCTATTACGCCAGC |
| SEQ ID NO: 3 | p2 | GTCCATGGTATATATCCTG\hat{ }CCAGCGGGCAGTGAGCGCAACGC |
| SEQ ID NO: 4 | p3 | TCTGCCAGTTCAGTTCGTTG |
| SEQ ID NO: 5 | p4 | TGCTGTCGGCTTTAACCTC |
| SEQ ID NO: 6 | p5 | GTCTCGGTCTCGATCTTTGG |
| SEQ ID NO: 7 | p6 | AGACCGGCAACAGGATTCAATC |
| SEQ ID NO: 8 | p7 | GCGGGCAGTGAGCGCAACGC |
| SEQ ID NO: 9 | p8 | GACCTCGAGTATGCTAGCTAC |
| SEQ ID NO: 10 | p9 | ATAACAATTTCACACAGGAAACAGCTATGAC |
| SEQ ID NO: 11 | p10 | ATCGTGGATAGCACTTTGGG |
| SEQ ID NO: 12 | p11 | TAAAAGGTGGCCCAAAGTGA |
| SEQ ID NO: 13 | p12 | CAAAAAAGCTCCGCACGAGGC |
| SEQ ID NO: 14 | p13 | CCCAAAGTGCTATCCACGAT |
| SEQ ID NO: 15 | p14 | TGCGCGCTATATTTTGTTTTC |
| SEQ ID NO: 16 | p15 | AGGGATCTAGTAACATAGATGACACCG |
| SEQ ID NO: 17 | p16 | CCAGTGAGCGCGCGTAATACG |
| SEQ ID NO: 18 | p17 | CTCTTCGCTATTACGCCAGC |
| SEQ 1D NO: 19 | p18 | TGCTGTCGGCTTTAACCTCT |
| SEQ ID NO: 20 | p19 | GATTGGTGGCATTGGAAC |
| SEQ ID NO: 21 | p20 | GATGACACCAACAGCCACAG |

Sequences of primers used in PCR reactions;
(i) Right border (RB) core sequences are underlined;
(ii) VirD2 cleavage sites are indicated by the “\hat{ }” symbol;
(iii) NcoI recognition sequence is indicated in italic font.

The PCR reaction mixture (25 μL) contained GC buffer, 0.2 mM dNTPs, 0.5 μM of each primer, 5 ng of DNA template and 0.5 U of high fidelity Phusion® DNA polymerase (Phusion is a registered trademark of Finnzymes Oy, Vantaa, Finland; the Phusion® DNA polymerase product was obtained from Fermentas Canada Inc., Burlington, ON, Canada). The amplification reactions consisted of a preliminary denaturation step at 98° C. for 30 s, followed by 10 cycles of 98° C. for 10 s, 59° C. for 30 s, 72° C. for 75 s, 20 cycles of 98° C. for 10 s, 67° C. for 30 s, 72° C. for 75 s followed by incubation at 72° C. for 10 min. The PCR product was extracted from agarose gel, purified using a QiaQuick® gel extraction kit (QuiQuick is a registered trademark of Qiagen GMBH, Hilden, Fed. Rep. Germany; the product was purchased from Qiagen Inc., Toronto, ON, Canada) and cloned into the NcoI site of LITMUS29 plasmid vector resulting in LITMUS29_iRB_P_{Act}-GUS-Tₙₒₜ_{_{—}}RB recombinant vector. T-DNA (4.8 kb long iRB_P_{Act}-GUS-Tₙₒₜ_{_{—}}RB cassette) was released from the recombinant vector by digestion with NcoI restrictase and extracted from agarose gel using a QiaQuick® gel extraction kit (Qiagen Inc.). This dsT-DNA was converted into the ssDNA form by heat denaturation for 10 min at 95° C. and immediate cooling on ice.

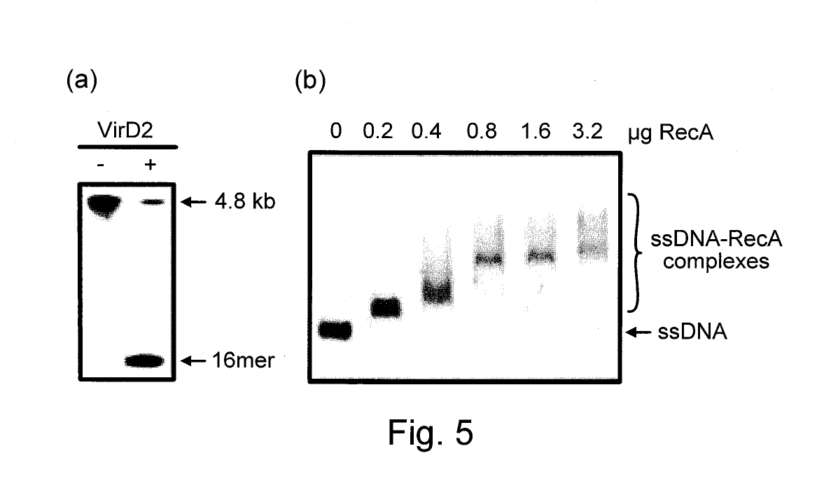

The VirD2-T-DNA complex was prepared as follows. The purified VirD2 protein was first tested for its cleavage activity using model oligonucleotides containing the RB sequence, following the method taught by Ziemienowicz et al. (2000, Mol Cell Biol. 20: 6317-6322). Optimization of the reaction was also performed by testing different salts and their concentration as well as various protein-to-oligonucleotide ratios. Under the most optimal conditions, the efficiency of the cleavage reaction was nearly 90% when 5-10 μg of VirD2 was used per 1 pmol of oligonucleotide. The VirD2-ssT-DNA complex was formed by reacting 2.0 μg of ssT-DNA with 10 μg of the VirD2 protein in TKM buffer (50 mM Tris-Cl pH 8.0, 150 mM KCl, 1 mM MgCl₂) for 1 h at 37° C. Cleavage efficiency of VirD2 on ssT-DNA was ˜75%, and ssDNA binding efficiency of RecA was 100% (FIG. 5).

Next, oligonucleotides used as primers for production of T-DNA (4.8 kb long NcoI/RB Act_GUS_nos_RB/NcoI cassette) by PCR were tested as the substrates for VirD2. The efficiency of the cleavage reaction was slightly lower than in the case of model oligonucleotide substrates, but still very high: 70-80% at the same protein:oligonucleotide ratio. Then, ssT-DNA (4.8 kb long RB Act_GUS_nos_RB cassette) was used as the substrate for VirD2. Cleavage efficiency of 75% was achieved by using 250 ng of VirD2 for 100 ng of DNA. The VirD2-T-DNA complex was then formed by reacting 2.0 μg of ssT-DNA with 10 μg of the VirD2 protein in TKM buffer (50 mM Tris-Cl pH 8.0, 150 mM KCl, 1 mM MgCl₂) for 1 h at 37° C.

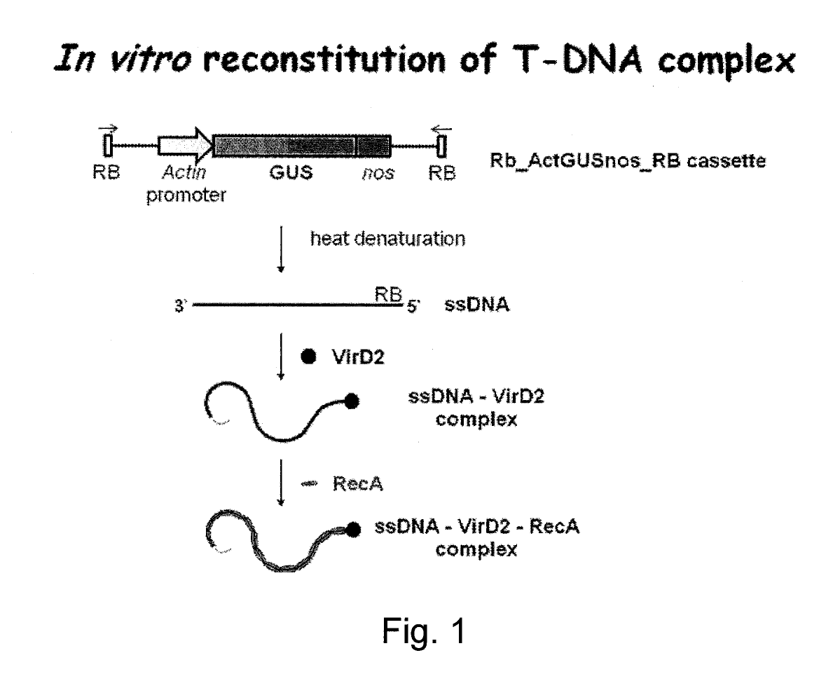

The VirD2-T-DNA-RecA complex was prepared as follows and illustrated in FIG. 1. VirD2-T-DNA complex (containing 4.8 kb long RB Act_GUS_nos_RB cassette) was reacted with an excess of E. coli RecA. 16 μg of the protein was reacted with 2.0 μg of ssT-DNA in complex with VirD2 during 30 min incubation at 37° C. following the method taught by Ziemienowicz et al. (2001, Plant Cell 13: 369-383).

The VirD2-T-DNA-RecA complex was then treated with CPP Tat2 following the method taught by Chugh et al. (2008, Study of uptake of cell penetrating peptides and their cargoes in permeabilized wheat immature embryos. FEBS J. 275(10): 2403-2414). Tat2 peptide was added to the formed VirD2-T-DNA-RecA complex at the ratio of 4:1 (4 μg of peptide per 1 μg of DNA). The two components were mixed and incubated for 15 minutes at room temperature. Next, 5 μg of lipofectamine were added followed by incubation for 5 minutes at the same conditions, and the reaction efficiency monitored following the method taught by Ziemienowicz et al. (1999, Proc. Natl. Acad. Sci. USA 96: 3729-3733).

### Example 2

### Transfection of Triticale Microspores with a T-DNA/Protein Nano-Complex Treated with the CPP Peptide Tat2

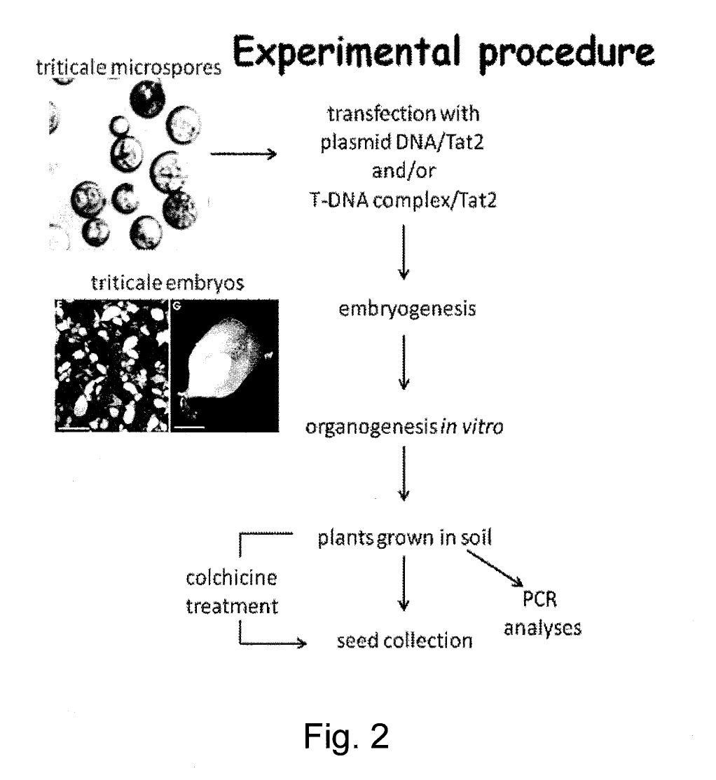

Triticale var. Ultima microspores were transfected with DNA or T-DNA/protein complexes in the presence or absence of the CPP Tat₂ peptide (SEQ ID NO: 1) following the steps outlined in FIG. 2. The reconstituted full T-DNA/protein complex (VirD2-ssT-DNA-RecA), partial complexes (ssT-DNA-RecA, VirD2-ssT-DNA) and naked DNA (ssT-DNA, dsT-DNA and pACT-1D/PstI) were used as carriers of the GUS gene in transfection experiments. Plasmid pACT-1D (pAct-1GUS), linearized with PstI restrictase, was used as a positive control. Tat₂ peptide to DNA ratio in all experiments was 4:1 (wt:wt). An additional step of treatment of the DNA-Tat₂ complexes with 5 mg of lipofectamin for 15 min was introduced prior to the transfection step. Four separate experiments were conducted with treatments having the Tat₂ peptide. Three separate experiments were conducted with treatments that did not receive the Tat₂ peptide.

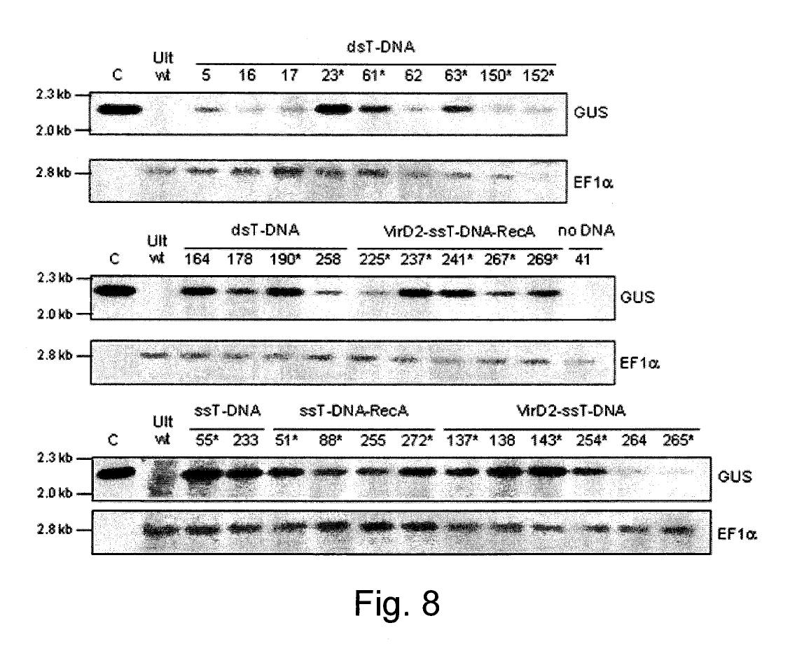

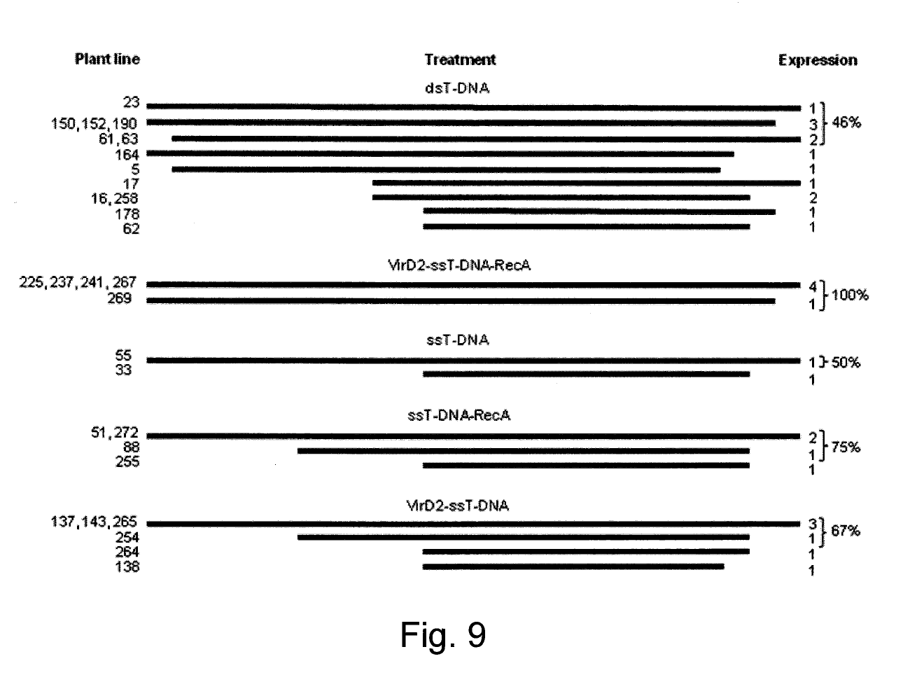

Transfected microspores were cultured in 30-mm Petri dishes containing liquid NPB-99 medium supplemented with 10% Ficoll in the presence of 4 ovaries per plate. Plates were incubated for 4-6 weeks at 28.5° C. in the dark. Formed embryos that were 1-2 mm long (FIG. 8), were transferred germ-side up to standard Petri dishes containing solid GEM medium and were incubated in a growth chamber with 16 hours light/8 hours dark photoperiod at 16° C. with light intensity of 80 μM/m²/s¹. Most embryos were not able to undergo any type of organogenesis and died, while some embryos generated roots only or alternatively albino plantlets (FIG. 9). A few embryos regenerated into green plantlets (FIG. 9, Table 2). Green plantlets were then transferred to root trainers containing soil-less growing mix and were cultivated in a green house.

Efficiency of regeneration of green plantlets varied between various samples from different treatments in all of the independent experiments as indicated by high standard deviation values. The average regeneration efficiency values did not exceed 10% in most cases, with the lowest value observed for transfection was with the full T-DNA/protein complex (VirD2-ssT-DNA-RecA) among all treatments in the presence of the Tat₂ CPP (Table 1). In total, 303 plants were regenerated in vitro and transplanted into soil-less growing mix. The survival rate of the transplanted plantlets was 93%, which resulted in 281 plants successfully cultured in the soil-less growing mix (Table 2).

The presence of the GUS transgene in genomes of plants regenerated after transfection was determined by PCR analysis and confirmed by Southern blot analysis. Genomic DNA was isolated from 100 mg leaf samples using the cetyltrimethylammonium bromide (CTAB) method disclosed by Doyle et al. (1987, Preservation of plant samples for DNA restriction endonuclease analysis. Taxon 36: 715-772), modified according to the DArT protocol (http://www.diversityarrays.com/sites/default/files/pub/DArT_DNA_isolation.pdf). To detect the GUS gene using PCR methodology, GUS specific primers p3 and p4 (SEQ ID NO: 4 and SEQ ID NO: 5 respectively) were combined with Actin intron p5 specific primer (SEQ ID NO: 6) and nos terminator p6 specific primer (SEQ ID NO: 7) (Table 1; FIG. 4). PCR reactions were performed in 25 μL reaction mixtures containing 1×CL buffer, 0.2 mM dNTPs, 0.1 μM of each primer, 0.025 U of Taq polymerase (Qiagen Inc.) and 100 ng of genomic DNA. The amplification reactions consisted of a preliminary denaturation step at 94° C. for 3 min, followed by 35 cycles of 94° C. for 1 min, 58° C. for 1 min, 72° C. for 1-1.5 min, and incubation at 72° C. for 10 min. PCR products were analyzed by electrophoresis in 0.8% agarose/1×TAE gel containing ethidium bromide at the concentration of 0.5 μg/mL.

TABLE 2
Plant regeneration from embryos obtained from control (untreated) microspores and from microspores treated with
various DNAs and DNA/protein nano-complexes in the presence or absence of the Tat₂ peptide. Numbers in the Table
represent average values (±SD) from four independent experiments with Tat₂ and three independent experiments
without Tat₂ .

| | Embryos | Abortive | Rooted | Albino | Green | Regeneration |
|---|---|---|---|---|---|---|
| Treatment | plated | embryos | embryos | plantlets | plantlets | efficiency |
| No treatment | 90.8 ± 7.4 | 49.3 ± 14.2 | 31.0 ± 14.4 | 4.3 ± 3.0 | 6.0 ± 2.8 | 6.6 ± 3.0% |
| No DNA + Tat₂ | 113.3 ± 24.8 | 73.8 ± 19.7 | 27.5 ± 16.3 | 5.3 ± 2.6 | 6.8 ± 6.1 | 6.1 ± 5.7% |
| pACT-1D/PstI + Tat₂ | 140.8 ± 39.5 | 71.3 ± 17.9 | 45.5 ± 21.0 | 10.3 ± 5.6 | 16.3 ± 14.2 | 11.5 ± 10.5% |
| dsT-DNA + Tat₂ | 163.0 ± 3.5 | 88.8 ± 19.3 | 46.3 ± 11.8 | 12.5 ± 4.4 | 9.5 ± 1.3 | 5.9 ± 0.9% |
| ssT-DNA + Tat₂ | 85.5 ± 36 6 | 35.3 ± 26.4 | 37.3 ± 4.8 | 7.5 ± 1.9 | 7.8 ± 7.6 | 8.3 ± 6.2% |
| ssT-DNA-RecA + Tat₂ | 126.8 ± 35.3 | 73.8 ± 37.8 | 31.8 ± 2.6 | 12.5 ± 4.5 | 6.3 ± 1.0 | 5.4 ± 1.9% |
| VirD2-T-DNA + Tat₂ | 126.3 ± 42.6 | 82.0 ± 36.2 | 33.0 ± 6.4 | 5.0 ± 6.0 | 6.3 ± 3.0 | 4.9 ± 1.4% |
| VirD2-T-DNA-RecA + Tat₂ | 173.0 ± 73.1 | 113.0 ± 40.7 | 33.0 ± 20.7 | 21.5 ± 16.7 | 5.3 ± 3.6 | 3.0 ± 1.5% |
| No DNA | 107.7 ± 42.4 | 74.3 ± 34.0 | 18.7 ± 17.7 | 9.7 ± 15.9 | 5.0 ± 6.1 | 6.6 ± 9.4% |
| pACT-1D/PstI | 109.7 ± 34.5 | 79.7 ± 14.0 | 22.0 ± 24.3 | 5.0 ± 4.6 | 3.0 ± 2.6 | 2.5 ± 2.2% |
| dsT-DNA | 74.3 ± 6.7 | 59.7 ± 11.0 | 5.3 ± 2.1 | 8.0 ± 6.2 | 1.3 ± 2.3 | 1.8 ± 3.1% |
| VirD2-ssT-DNA-RecA | 70.7 ± 20.2 | 50.0 ± 6.6 | 12.7 ± 11.0 | 4.7 ± 7.1 | 3.3 ± 5.8 | 3.5 ± 6.1% |

TABLE 3
Analysis of the intactness of the GUS expression cassette in plants regenerated from triticale microspores.

| | Plants containing the amplified regions of the transgene source DNA | | | | | | | | | |
|---|---|---|---|---|---|---|---|---|---|---|
| | P1-RS | P1-Act | Act-GUS | Act-GUS | Act-GUS | GUS-nos | GUS-nos | GUS-nos | GUS-P2 | GUS-P2 |
| Treatment | p7-p8 | p9-p10 | p11-p12 | p13-p3 | p5-p3 | p4-p6 | p4-p14 | p4-p15 | p4-p16 | p4-p17 |
| No treatment (control | 0 | 0 | 0 | 0 | 0 | 0 | 0 | 0 | 0 | 0 |
| (21 plants) | 0% | 0% | 0% | 0% | 0% | 0% | 0% | 0% | 0% | 0% |
| No DNA + Tat₂ | 0 | 0 | 0 | 0 | 1 | 2 | 2 | 2 | 0 | 0 |
| (26 plants) | 0% | 0% | 0% | 0% | 3.9% | 7.7% | 7.7% | 7.7% | 0% | 0% |
| pACT-1D/PstI + Tat₂ | 0 | 0 | 12 | 14 | 14 | 14 | 14 | 14 | 14 | 14 |
| (63 plants) | 0% | 0% | 19.1% | 22.2% | 22.2% | 22.2% | 22.2% | 22.2% | 22.2% | 22.2% |
| dsT-DNA + Tat₂ | 5 | 7 | 9 | 10 | 13 | 13 | 13 | 11 | 6 | 4 |
| (44 plants) | 11.4% | 15.9% | 20.5% | 22.7% | 29.6% | 29.6% | 29.6% | 25.0% | 13.6% | 9.1% |
| ssT-DNA + Tat₂ | 1 | 1 | 1 | 1 | 2 | 2 | 2 | 2 | 1 | 1 |
| (27 plants) | 3.7% | 3.7% | 3.7% | 3.7% | 7.4% | 7.4% | 7.4% | 7.4% | 3.7% | 3.7% |
| ssT-DNA-RecA + Tat₂ | 2 | 2 | 3 | 3 | 4 | 4 | 4 | 4 | 2 | 2 |
| (23 plants) | 8.7% | 8.7% | 13.0% | 13.0% | 17.4% | 17.4% | 17.4% | 17.4% | 8.7% | 8.7% |
| VirD2-ssT-DNA + Tat₂ | 3 | 3 | 4 | 5 | 6 | 6 | 5 | 4 | 3 | 3 |
| (23 plants) | 13.0% | 13.0% | 17.4% | 21.7% | 26.1% | 26.1% | 21.7% | 17.4% | 13.0% | 13.0% |
| VirD2-ssT-DNA-RecA + Tat₂ | 5 | 5 | 5 | 5 | 5 | 5 | 5 | 5 | 5 | 4 |
| (20 plants) | 25.0% | 25.0% | 25.0% | 25.0% | 25.0% | 25.0% | 25.0% | 25.0% | 25.0% | 20% |
| No DNA | 0 | 0 | 0 | 0 | 0 | 0 | 0 | 0 | 0 | 0 |
| (14 plants) | 0% | 0% | 0% | 0% | 0% | 0% | 0% | 0% | 0% | 0% |
| pACT-1D/PstI | 0 | 0 | 0 | 0 | 1 | 1 | 1 | 1 | 0 | 0 |
| (9 plants) | 0% | 0% | 0% | 0% | 11.1% | 11.1% | 11.1% | 11.1% | 0% | 0% |
| dsT-DNA | 0 | 0 | 0 | 0 | 0 | 0 | 0 | 0 | 0 | 0 |
| (3 plants) | 0% | 0% | 0% | 0% | 0% | 0% | 0% | 0% | 0% | 0% |
| VirD2-ssT-DNA-RecA | 0 | 0 | 0 | 0 | 0 | 0 | 0 | 0 | 0 | 0 |
| (10 plants) | 0% | 0% | 0% | 0% | 0% | 0% | 0% | 0% | 0% | 0% |

PCR analysis revealed that omission of the Tat2 peptide from the nano-complex resulted in low transfection efficiency evidenced by no or very few GUS-positive plants (Table 3). In contrast, DNA and DNA-protein complexes were transfected efficiently into triticale microspores via Tat₂ peptide (Table 3). The percentage of GUS-positive plants was comparable when complete T-DNA complex (VirD2-ssT-DNA-RecA), ssT-DNA-VirD2 or linear naked dsDNA (pACT-1D/PstI, dsTDNA) were used for transfection (Table 2). Slightly lower values were observed for the ssT-DNA-RecA complex, whereas use of naked ssT-DNA generated low number of GUS-positive plants (Table 3), most likely due to the lack or incomplete protection of DNA from nucleases. Among naked DNA molecules, dsDNA was protected from nucleolytic degradation better than ssDNA (Table 3). All the control untreated plants were GUS-negative and only very few GUS-positive plants were found among those treated without DNA (Table 3). The latter ones likely represent false positive cases.

Next, Southern blot analysis was performed to verify PCR results. gDNA from GUS-positive triticale lines was digested with BamHI and XbaI and probed first with the GUS-specific probe and then with a probe specific for wheat EF1α gene. Transgenic and non-transformed triticale genomic DNA was isolated as described above and treated with RNaseA (final concentration: 80 μg/mL) for 10 min at 65° C., followed by purification using the phenol-chloroform method and precipitation with ethanol. gDNA was then digested using restriction enzymes: (a) BamHI and XbaI to test for the transgene presence, and (b) BamHI alone to test for the transgene copy number in a 500-μL reaction mixture containing NEB#3 buffer, 1 mg/mL BSA, 30 μg of gDNA and 400 U of the restrictase. The reactions were incubated at 37° C. over night. Digested DNA was purified using the phenol-chloroform method and concentrated by precipitation with ethanol. Southern blot analysis was performed following a modification to the protocol taught by Sambrook et al. (2001, Molecular Cloning: a Laboratory Manual, 3^{rd} ed. Cold Spring Harbor Laboratory Press, Cold Spring Harbor N.Y., USA). Twenty μg of digested gDNA were separated on a 0.8% agarose gel at 35V for 16 h in 1×TAE buffer. The gel was rinsed in dH₂O, de-purinated for 15 min in 0.25N HCl, rinsed in dH₂O, denatured for 30 min in 0.4N NaOH, rinsed again in dH₂O, neutralized for 15 min in 0.5 M Tris-HCl pH 7.5 containing 3 M NaCl and soaked in transfer buffer (10×SSC: 1.5 M NaCl, 0.15 M sodium citrate) for 10 min. DNA transfer onto a positively charged nylon membrane was performed for 2.5 h using a vacuum blotter. DNA was then cross-linked to the positively charged nylon membrane at 120 mJ/cm² in Spectrolinker Crosslinker (Spectromics Corp., Westbury, N.Y., USA). Probes were prepared using a PCR DIG Probe Synthesis kit (Roche Diagnostics, Laval, QC, Canada) and following to the protocol provided by the supplier. p18 and p6 primers were used for the GUS-specific probe and p19 and p20 primers were used for the EF1α-specific probe. Hybridization was carried out using DIG Easy Hyb solution (Roche Diagnostics) at 42° C. (GUS probe) or 65° C. (EF1α probe). Detection was performed using AP-conjugated anti-DIG antibodies (Roche Diagnostics) diluted 1:2,500 in blocking solution containing 1% Blocking Reagent (Roche Diagnostics) in maleic acid buffer (0.1 M maleic acid, 0.5 M NaCl, pH 7.5) and CPD Star (Roche Diagnostics) as a substrate. Images of the membrane were taken with Fluor Chem HD2 (Convergent Bioscience, Toronto, ON, Canada).

Presence of the GUS gene was confirmed in all transformed lines but not in Ultima wild type plants and plant lines regenerated from microspores transfected without DNA (e.g., line #41; FIG. 8). Since this line was found to be GUS-positive by the PCR method, the latter result indicates that this line represents a false-positive case. The intensity of the Southern blot signal detected by the GUS-specific probe varied between the lines, whereas the intensity of the endogenous control (EF1α gene) was constant in all lines (FIG. 8). These results suggested variations in the transgene copy numbers.

Next, GUS-positive plants were analyzed for the intactness of the integrated T-DNA. Analysis of the intactness of the integrated transgene cassette was performed by PCR using ten sets of primers specific to various regions of the GUS cassette (FIG. 4; Table 3). The analysis revealed that when naked dsT-DNA or ssT-DNA or ssT-DNA combined with RecA or VirD2 protein and Tat₂ peptide were used to transfect triticale microspores, some truncations of either 5′, 3′ or of both ends of the transgene cassette were observed (FIG. 9; Table 3). In contrast, the use of the complete nano-complex (VirD2-ssT-DNA-RecA+Tat₂) resulted in nearly 100% intact integration events, with only one case of short truncation of the 3′ end (FIG. 9; Table 3). According to our data, about 50% of the GUS-positive plants obtained after Tat₂-mediated transfection of triticale microspores with naked dsT-DNA or ssT-DNA were expected to be expressed (FIG. 9). In the case of plants from transfection experiment with ssT-DNA complexed with either RecA or VirD2 protein alone (+Tat₂), the percentage of GUS-positive plants that express the transgene could be higher: 75% and 67%, respectively. The highest percentage number (100%) was expected for GUS-positive plants regenerated from microspores transfected with the complete nano-complex (VirD2-ssT-DNA-RecA+Tat₂), as they contain complete regulatory elements (promoter and terminator) required for the normal transgene expression.

Analysis of transgene expression was performed at the protein level using Western blotting technique on crude extracts from GUS-positive plants. GUS transgene was expected to be expressed in lines #23, 61, 63, 152 and 190 (from treatment with dsT-DNA+Tat₂), 55 (ssT-DNA+Tat₂ treatment), 51, 88 and 272 (ssT-DNA-RecA+Tat₂ treatment), 137, 143, 254 and 265 (VirD2-T-DNA+Tat₂ treatment) as well as 225, 237, 241, 267 and 269 (VirD2-T-DNA-RecA+Tat₂ treatment). Crude protein extracts were prepared from leaf tissue (100 mg) following the method taught by Stoger et al. (1999, Expression of the insecticidal lectin from snowdrop (Galanthus nivalis agglutinin; GNA) in transgenic wheat plants: effect on predation by the grain aphid Sitobion avenae. Mol. Breed. 5: 65-73) using extraction buffer supplemented with Complete Protease Inhibitor Cocktail (Roche Diagnostics). Aliquots of 10 μg of total protein were analyzed by Western blotting in following a standard protocol disclosed by Sambrook et al. (2001) using polyclonal rabbit antibodies raised against the N-terminal peptide of bacterial β-glucuronidase (1:2,000; primary antibody; Abcam, Cambridge, Mass., USA) and donkey antibody to rabbit IgG (HRP conjugate; 1:10,000; secondary antibody; Abcam). Detection was carried out with ECL Plus Western blotting detection reagents according to the manufacturer's recommendations (GE Healthcare Biosciences, Uppsala, Sweden). Membranes were then exposed to X-ray films and the intensity of the signals was quantified using ImageJ software.

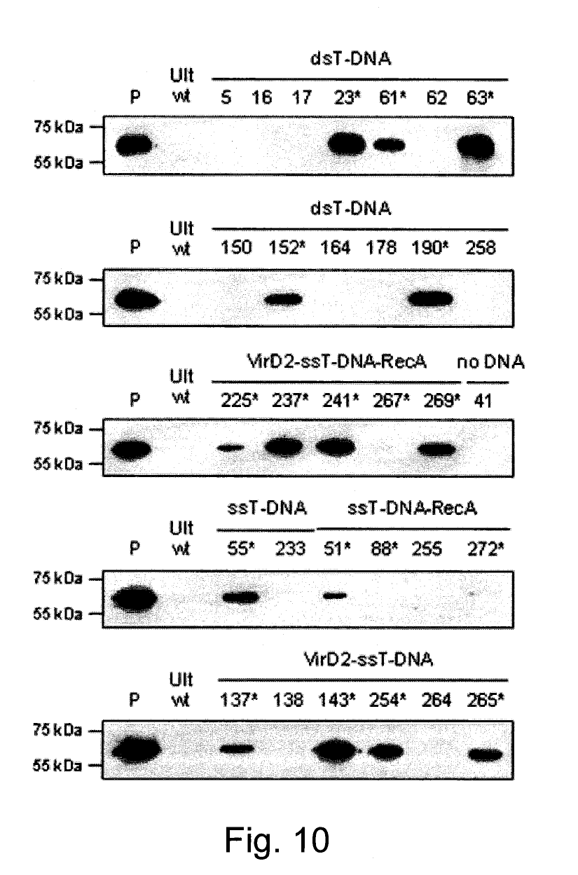

Results of the Western blot analysis showed that the bacterial β-glucuronidase protein was detected in all lines predicted to express the transgene according to PCR and Southern blot analyses (FIG. 10 and Table 4) with one line (#88) expressing the GUS transgene at extremely low level. Line #88 most likely represents the case of GUS-positive plant carrying incomplete P_{Act}-GUS-Lₙₒₛ cassette that lacks <100 by of the promoter's 5′ end. We also tested some GUS-positive plants that were predicted not to express the GUS gene due to larger truncations of the transgene cassette (line #5, 16, 17, 62, 150, 164, 178, 258, 233, 252, 138 and 264) or due to the absence of the transgene (line #41), and indeed no GUS protein was detected in these lines (FIG. 10). Significant differences in the β-glucuronidase protein level were observed in GUS positive plants from all treatments with T-DNA and T-DNA/protein complexes (+Tat₂) with some levels of the GUS protein being very high (FIG. 10). These differences may reflect variations in the transgene copy number.

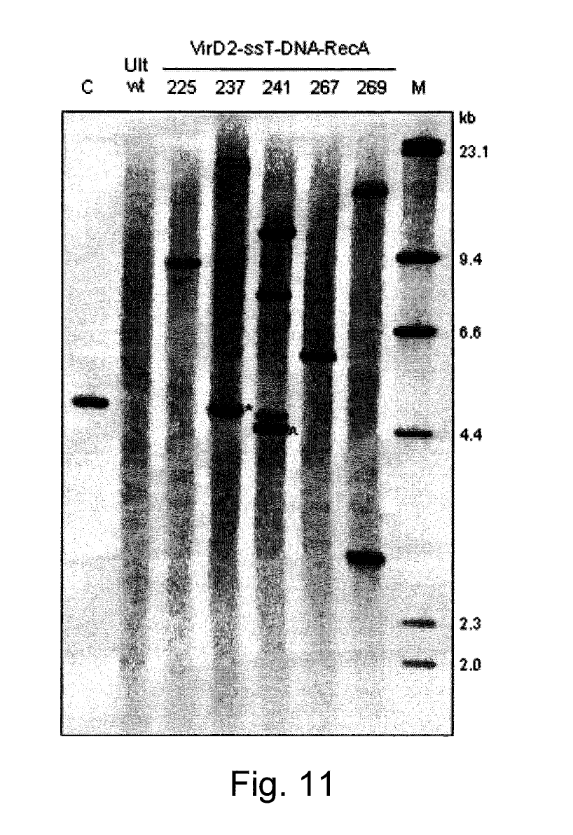

It is known that standard procedures of plant transformation using Agrobacterium often result in clear integration patterns exemplified by low number of copies of integrated transgenes (Windels et al., 2008, Agrobacterium tumefaciens-mediated transformation: patterns of T-DNA integration into the host genome. In: Tzfira et al. (Eds.) Agrobacterium: from biology to biotechnology pp 442-483), whereas most other methods used in plant biotechnology such as direct gene transfer, bombardment, and the like, result in integration of multiple DNA molecules and, as consequence, multiple copy/multiple loci insertion patters, that may lead to variations in the transgene expression (Latham et al., 2006, The mutational consequences of plant transformation. J. Biomed. Biotech. 25376: 1-7). The transgene copy number and integration pattern were analyzed by Southern blotting using gDNA of transgenic plants expressing the transgene at the detectable level. gDNA was digested with BamHI and hybridized with the GUS-specific probe. Application of the CPP-mediated transgene delivery resulted in low copy number (≦5) and relatively simple patterns of transgene integration (FIG. 11; Table 4). However, transgenic plants regenerated from microspores transfected with dsT-DNA+Tat₂ were found to contain 1.6-fold higher transgene copy number and 1.4-fold higher integration locus number than plants obtained by transfection with the VirD2-ssT-DNA-RecA+Tat₂ nano-complex. These values are actually higher, as the 2-fold difference in the number of DNA molecules in the transfection samples should be also taken into consideration. More importantly, 40% of the nano-complex-transfected plants showed a single copy single locus integration pattern (FIG. 11; Table 4), which was not observed in plants transfected with dsT-DNA (Table 4). In addition, the latter plants showed more frequent events of integration of at least two DNA molecules into a single locus, such as head-to-head and head-to-tail or tail-to-head integrations (Table 4).

Finally, the transgene copy number and expression level were compared. The comparison revealed, in most cases, a clear correlation between these two factors (Table 4). Two lines containing a single copy of the transgene (line #267 and 88) showed very low protein levels. In the instance of another line (#272), low level of GUS expression is most likely caused by gene silencing induced by additional copies of the transgene. Variations in the transgene protein level were noted also in plants regenerated from microspores transfected with the complete nano-complex (VirD2-ssT-DNA-RecA+Tat₂; FIG. 10; Table 4). Such variations in transgene expression are sometimes observed in plants generated by the classical Agrobacterium-mediated transformation, with most transformants expressing transgenes at relatively low levels (Filipecki et al., 2006, Unintended consequences of plant transformation: a molecular insight. J. Appl. Genet., 47: 277-286). Improvement of the expression pattern of transgenes should be possible in the future by changing the ratio of Tat₂ to T-DNA allowing the formation of smaller DNA-CPP complexes which will result in the delivery of fewer T-DNA molecules into plant cells.

TABLE 4
Comparison of the transgene copy number and transgene
expression in plants regenerated from microspores
transfected with various types of T-DNA and T-DNA/protein
complexes in the presence of Tat₂ CPP.

| Treat- | | | | |
|---|---|---|---|---|
| ment/ | | | Transgene | Integration |
| plant | Protein | BamHI fragment | copy | locus |
| line | amount | sizes (kb) | number | number |

dsT-DNA

| | | | | | | | |
|---|---|---|---|---|---|---|---|
| 23 | 50.3 ± 2.5 | 4.8 | 6.4 | 7.8 | | 3 | 3 |
| 61 | 29.0 ± 3.2 | 2.8 | ~20 | | | 2 | 2 |
| 63 | 50.7 ± 3.5 | 4.8* | 7.4 | 14.4 | | 4 | 3 |
| 152 | 30.6 ± 1.8 | 4.4\hat{ } | 4.8* | 10.8 | | 5 | 3 |
| 190 | 43.5 ± 2.2 | 4.8* | 5.4 | 5.6 | 7.6 | 5 | 4 |

| | | |
|---|---|---|
| Means: | 3.8 ± 1.3 | 3.0 ± 0.7 |

VirD2-ssT-DNA-VirE2

| | | | | | | | |
|---|---|---|---|---|---|---|---|
| 225 | 12.3 ± 1.2 | 8.8 | | | | 1 | 1 |
| 237 | 45.7 ± 3.4 | 4.8* | 19.2 | | | 3 | 2 |
| 241 | 43.5 ± 2.2 | 4.4\hat{ } | 4.5 | 7.4 | 12.0 | 5 | 4 |
| 267 | 2.5 ± 0.5 | 5.8 | | | | 1 | 1 |
| 269 | 36.6 ± 2.1 | 2.6 | 15.0 | | | 2 | 2 |

| | | |
|---|---|---|
| Means: | 2.4 ± 1.7 | 2.0 ± 1.2 |

ssT-DNA

| | | | | | |
|---|---|---|---|---|---|
| 55 | 28.3 ± 2.6 | 5.3 | 9.4 | 2 | 2 |

ssT-DNA-RecA

| | | | | | | |
|---|---|---|---|---|---|---|
| 51 | 11.1 ± 0.8 | 11.0 | | | 1 | 1 |
| 88 | 0.8 ± 0.2 | 2.8 | | | 1 | 1 |
| 272 | 1.3 ± 1.9 | 4.2 | 4.8* | 7.2 | 4 | 3 |

| | | |
|---|---|---|
| Means: | 2.0 ± 1.7 | 1.7 ± 1.2 |

VirD2-ssT-DNA

| | | | | | | |
|---|---|---|---|---|---|---|
| 137 | 17.1 ± 1.5 | 9.6 | | | 1 | 1 |
| 143 | 40.6 ± 3.5 | 4.8 | 10.8 | | 2 | 2 |
| 254 | 27.5 ± 1.8 | 2.7 | 6.0 | 14.8 | 3 | 3 |
| 265 | 22.3 ± 2.1 | 5.8 | 8.8 | | 2 | 2 |

| | | |
|---|---|---|
| Means: | 2.0 ± 0.8 | 2.0 ± 0.8 |

Head-to-tail or tail-to-head integrations (4.8 kb) are indicated with asterisks whereas head-to-head integrations (4.4 kb) are marked by a \hat{ } symbol.

In summary, we developed novel T-DNA/protein nano-complexes and novel methods for the uses thereof for plant transformation. Furthermore, we have shown that these T-DNA/protein nano-complexes and related methods of use are suitable for plant species that are difficult to transform with the classical Agrobacterium-mediated techniques. Moreover, CPP-mediated delivery of the T-DNA complex results in Agrobacterium-like type of transgene integration pattern regarding transgene intactness, copy numbers generated, and expression efficiency. Moreover, our T-DNA/protein nano-complex strategy yields more frequent integration of intact transgene molecules into a single locus of a monocot genome resulting in efficient expression of the transgene in transgenic plants.

1. A DNA/protein nano-complex comprising:
an expressible nucleotide molecule complexed with a VirD2 protein molecule; and
a RecA protein molecule complexed with the nucleotide molecule/VirD2 protein molecule complex.

2. The DNA/protein nano-complex of claim 1, additionally comprising a cell penetrating peptide complexed with the nucleotide molecule/VirD2 protein molecule/RecA protein molecule complex.

3. The DNA/protein nano-complex of claim 2, wherein the cell penetrating peptide comprises an amino acid sequence that shares at least 75% sequence identity with a Tat2 protein.

4. The DNA/protein nano-complex of claim 2, wherein the cell penetrating peptide comprises an amino acid sequence that shares at least 75% sequence identity with SEQ NO ID: 1.

5. The DNA/protein nano-complex of claim 2, wherein the cell penetrating peptide comprises an amino acid sequence that is SEQ ID NO: 1.

6. The DNA/protein nano-complex of claim 1, wherein the expressible nucleotide molecule is operably linked to a carrier plasmid.

7. The DNA/protein nano-complex of claim 6, wherein the carrier plasmid is a pACT-1D plasmid.

8. The DNA/protein nano-complex of claim 6, wherein the carrier plasmid comprises a right border sequence from an Agrobacterium pTi plasmid.

9. The DNA/protein nano-complex of claim 1, wherein the expressible nucleotide molecule comprises a single strand.

10. The DNA/protein nano-complex of claim 1, wherein the expressible nucleotide molecule comprises a double strand.

11. The DNA/protein nano-complex of claim 1, wherein the expressible nucleotide molecule is operably linked to a promoter.

12. The DNA/protein nano-complex of claim 1, wherein the expressible nucleotide molecule comprises a sequence that encodes for a protein or proteins that would confer a transgenic plant with herbicide resistance or tolerance; insect resistance or tolerance; disease resistance or tolerance; stress tolerance or resistance; increased yield; food content and makeup; physical appearance; male sterility; drydown; standability; prolificacy; starch properties; oil quantity and quality; or a combination thereof.

13. The DNA/protein nano-complex of claim 1, wherein the expressible nucleotide molecule is covalently linked to the VirD2 protein molecule.

14. The DNA/protein nano-complex of claim 1, wherein the VirD2 protein molecule comprises an amino acid sequence that shares at least 75% sequence identity with SEQ ID NO: 22.

15. The DNA/protein nano-complex of claim 1, wherein the VirD2 protein molecule comprises an amino acid sequence that is SEQ ID NO: 22.

16. The DNA/protein nano-complex of claim 1, wherein the RecA protein molecule comprises an amino acid sequence that shares at least 75% sequence identity with SEQ ID NO: 23.

17. The DNA/protein nano-complex of claim 1, wherein the RecA protein molecule comprises an amino acid sequence that is SEQ ID NO: 23.

18. A method for preparing a DNA/protein nano-complex, comprising the steps of:
preparing a DNA construct comprising an expressible nucleotide molecule operably linked with a VirD2 target sequence;
exposing the DNA construct to VirD2 protein molecules whereby the VirD2 target sequence is cleaved and a nucleotide molecule/VirD2 protein molecule complex is formed; and
exposing the nucleotide molecule/VirD2 protein molecule complex to a RecA protein molecule whereby a nucleotide molecule/VirD2 protein molecule/RecA protein molecule complex is formed.

19. The method of claim 18, additionally comprising a step of exposing the nucleotide molecule/VirD2 protein molecule/RecA protein molecule complex to a cell penetrating peptide (CPP) whereby a nucleotide molecule/VirD2 protein molecule/RecA protein molecule/CPP complex is formed.

20. The method of claim 19, wherein the cell penetrating peptide comprises an amino acid sequence that shares at least 75% sequence identity with a Tat2 protein.

21. The method of claim 19, wherein the cell penetrating peptide comprises an amino acid sequence that shares at least 75% sequence identity with SEQ NO ID: 1.

22. The method of claim 19, wherein the cell penetrating peptide comprises an amino acid sequence that is SEQ ID NO: 1.

23. Use of a DNA/protein nano-complex according to claim 1, to transfect a eukaryotic cell.

24. Use according to claim 23, wherein the eukaryotic cell is a plant cell.

25. Use according to claim 24, wherein the plant cell is a monocot plant cell.

26. Use according to claim 24, wherein the plant cell is a monocot plant cell selected from the group consisting of wheat cells, corn cells, triticale cells, barley cells, and rye cells.


SEQUENCE LISTING (23 sequences)

SEQ ID NO: 1 - protein, 18 aa, Unknown
  tat2 CPP peptide
RKKRRQRRRRKKRRQRRR

SEQ ID NO: 2 - DNA, 42 bp, Unknown
  p1 primer
agccatggtatatatcctgccactcttcgctattacgccagc

SEQ ID NO: 3 - DNA, 42 bp, Unknown
  p2 primer
gtccatggtatatatcctgccagcgggcagtgagcgcaacgc

SEQ ID NO: 4 - DNA, 20 bp, Unknown
  p3 primer
tctgccagttcagttcgttg

SEQ ID NO: 5 - DNA, 19 bp, Unknown
  p4 primer
tgctgtcggctttaacctc

SEQ ID NO: 6 - DNA, 20 bp, Unknown
  p5 primer
gtctcggtctcgatctttgg

SEQ ID NO: 7 - DNA, 22 bp, Unknown
  p6 primer
agaccggcaacaggattcaatc

SEQ ID NO: 8 - DNA, 20 bp, Unknown
  p7 primer
gcgggcagtgagcgcaacgc

SEQ ID NO: 9 - DNA, 21 bp, Unknown
  p7 primer
gacctcgagtatgctagctac

SEQ ID NO: 10 - DNA, 31 bp, Unknown
  p9 primer
ataacaatttcacacaggaaacagctatgac

SEQ ID NO: 11 - DNA, 20 bp, Unknown
  p10 primer
atcgtggatagcactttggg

SEQ ID NO: 12 - DNA, 20 bp, Unknown
  p11 primer
taaaaggtggcccaaagtga

SEQ ID NO: 13 - DNA, 21 bp, Unknown
  p12 primer
caaaaaagctccgcacgaggc

SEQ ID NO: 14 - DNA, 20 bp, Unknown
  p13 primer
cccaaagtgctatccacgat

SEQ ID NO: 15 - DNA, 21 bp, Unknown
  p14 primer
tgcgcgctatattttgttttc

SEQ ID NO: 16 - DNA, 27 bp, Unknown
  p15 primer
agggatctagtaacatagatgacaccg

SEQ ID NO: 17 - DNA, 21 bp, Unknown
  p16 primer
ccagtgagcgcgcgtaatacg

SEQ ID NO: 18 - DNA, 20 bp, Unknown
  p17 primer
ctcttcgctattacgccagc

SEQ ID NO: 19 - DNA, 20 bp, Unknown
  p18 primer
tgctgtcggctttaacctct

SEQ ID NO: 20 - DNA, 18 bp, Unknown
  p19 primer
gattggtggcattggaac

SEQ ID NO: 21 - DNA, 20 bp, Unknown
  p20 primer
gatgacaccaacagccacag

SEQ ID NO: 22 - protein, 447 aa, Unknown
  VirD2 protein
MPDRAQVIIRIVPGGGTKTLQQIINQLEYLSRKGRLELQRSARHLDIPLPPDQIHELARSWVQETGTYDESQPDEERQQELTTHIIVSFPAGTSQVAAYAASREWAAEMFGSGAGGGRYNYLTAFHIDRDHPHLHVVVNRRELLGHGWLKISRRHPQLNYDALRIKMAEISLRHGIALDASRRAERGITERPITYAQYRRLEREQARQIRFEDADLEQSSPQGDHPEFSQPFDTSPFEASAGGPEDMPRPNNRQNESQVHLQEPAGVSNEAGVLVRVALETERLAQPFVSETILADDIGSGSSRVAEGRVESANRTPDIPRAATEAATHTTHDRQRRAKRPHDDDGGPSGAKRVTLEGIAVGPQANAGEQDGSSGPLVRQAGTSRPSPPTATTRASTATDSLSATAHLQQRRGVLSKRPREDDDGEPSERKRERDERSKDGRGGNRR

SEQ ID NO: 23 - protein, 352 aa, Unknown
  RecA protein
MAIDENKQKALAAALGQIEKQFGKGSIMRLGEDRSMDVETISTGSLSLDIALGAGGLPMGRIVEIYGPESSGKTTLTLQVIAAAQREGKTCAFIDAEHALDPIYARKLGVDIDNLLCSQPDTGEQALEICDALARSGAVDVIVVDSVAALTPKAEIEGEIGDSHMGLAARMMSQAMRKLAGNLKQSNTLLIFINQIRMKIGVMFGNPETTTGGNALKFYASVRLDIRRIGAVKEGENVVGSETRVKVVKNIAAPFKQAEFQILYGEGINFYGELVDLGVKEKLIEKAGAWYSYKGEKIGQGKANATAWLKDNPETAKEIEKKVRELLLSNPNSTPDFSVDDSEGVAETNEDF